SEQUENCE LISTING (8 sequences)

SEQ ID NO: 1 - DNA, 22 bp, Artificial
  Synthetic oligonucleotide
ccacaattttaaaagaaaaggg

SEQ ID NO: 2 - DNA, 55 bp, Artificial
  Synthetic oligonucleotide
  promoter (1)..(26)
aatttaatacgactcactatagggagagtttgtatgtctgatgctattatgtcta

SEQ ID NO: 3 - RNA, 23 nt, Artificial
  Synthetic oligonucleotide
  modified_base (7)..(7): Inosine
  misc_feature (7)..(7): n is a, c, g, or u
cgggcgnggguacagugcgcccg

SEQ ID NO: 4 - DNA, 57 bp, Artificial
  Synthetic oligonucleotide
ucugcugucccuguaauaaacccgtttaaaaaaaaaaaaaaaaaaaaaaaaaaaaaa

SEQ ID NO: 5 - DNA, 21 bp, Artificial
  Synthetic oligonucleotide
ctatatactcaaggaagtgac

SEQ ID NO: 6 - DNA, 51 bp, Artificial
  Synthetic oligonucleotide
  Promoter (1)..(27)
aatttaatacgactcactatagggagagatgattgacttgtgattccgctc

SEQ ID NO: 7 - RNA, 26 nt, Artificial
  Synthetic oligonucleotide
ccgagcaaguguacuagccgacucgg

SEQ ID NO: 8 - DNA, 57 bp, Artificial
  Synthetic oligonucleotide
cguucacuauuggucucugcauuctttaaaaaaaaaaaaaaaaaaaaaaaaaaaaaa

(19) United States
(12) Patent Application Publication
(10) Pub. No.: US 2006/0286587 A1
(43) Pub. Date: Dec. 21, 2006

(54) Methods for quantitative analysis of a nucleic acid amplification reaction

(75) Inventors: Richard S. Lee, San Diego, CA (US); Michael M. Becker, San Diego, CA (US); Michael R. Reshatoff, San Diego, CA (US)

Correspondence Address: GEN PROBE INCORPORATED, 10210 GENETIC CENTER DRIVE, SAN DIEGO, CA, 92121, US

(73) Assignee: GEN-PROBE INCORPORATED, San Diego, CA (US)

(21) Appl. No.: 11/452,742

(22) Filed: Jun. 14, 2006

Related U.S. Application Data
(60) Provisional application No. 60/691,272, filed on Jun. 15, 2005.

(51) Int. Cl. C12Q 1/68; G06F 19/00

(52) U.S. Cl. 435/6; 702/20

(57) ABSTRACT

Methods for quantitating an initial amount of a target nucleic acid in a sample which has been subjected to in vitro nucleic acid amplification to produce data that is analyzed by using a Fourier Transform based algorithm are disclosed.

### RELATED APPLICATION

This application claims the benefit under 35 U.S.C. 119(e) of provisional application No. 60/691,272, filed Jun. 15, 2005, which is incorporated by reference herein.

### FIELD OF THE INVENTION

This invention relates to methods for quantifying nucleic acids, and more specifically relates to methods for determining a starting quantity of a nucleic acid sequence in a sample from amplified sequences in a nucleic acid amplification reaction, which may be associated with an apparatus or computerized device.

### BACKGROUND OF THE INVENTION

Nucleic acid amplification in vitro may be accomplished by using a variety of techniques to selectively make copies of a particular target nucleic acid sequence or its complement starting from a limited number of target sequences present in a sample. Known methods of nucleic acid amplification include, e.g., the polymerase chain reaction (PCR, e.g., described in U.S. Pat. Nos. 4,683,195, 4,683,202, and 4,800,159; Methods in Enzymology, 1987, Vol. 155: 335-350), Qβ-replicase mediated amplification (e.g., described in U.S. Pat. No. 4,786,600), the ligase chain reaction (LCR, e.g., described in EP Pat. App. No. 0 320 308), strand-displacement amplification (SDA, e.g., Walker et al., 1992, Proc. Natl. Acad. Sci. USA 89:392-396, and U.S. Pat. No. 5,422,252), and methods that rely on transcription of sequences, generally referred to as transcription-associated amplification (e.g., U.S. Pat. Nos. 5,399,491 and 5,554,516, Kacian et al., U.S. Pat. No. 5,437,990, Burg et al., PCT Pub. Nos. WO 88/01302 and WO 88/10315, Gingeras et al., U.S. Pat. No. 5,130,238. Malek et al., and U.S. Pat. Nos. 4,868,105 and 5,124,246, Urdea et al.). Some applications of nucleic acid amplification make additional copies of the sequence of interest and detect the amplified products or specific sequences, such as by detecting attachment of a DNA dye or a sequence-specific probe to the amplified products. Some applications perform additional manipulations on the amplified sequences, such as determining the sequence of the amplified products. Some applications quantitate the amount of the initial sequence of interest in the sample to provide diagnostic or prognotic information related to a biological agent or genetic element in a sample.

Various methods have been used in quantitative analysis of nucleic acid sequences. Some methods measure the level of amplified nucleic acids at the endpoint of an amplification reaction and use that value to determine the starting quantity or concentration of the target nucleic acid in the sample (e.g., Rodriguez et al., 1992, Nucl. Acids Res. 20: 3528, Zimmermann et al., 1996, BioTechniques 21: 280). Such methods often use an amplification factor related to the number of cycles of amplification that have occurred and the efficiency of replication in each cycle, which is related to the number of targets or amplified products in the amplification reaction. Some methods, often referred to a “real-time” detection, measure amplification products during the amplification reaction, usually during the exponential phase of the growth curve, which may be performed with or without an external or internal standard, to determine an initial quantity of the target in a sample (e.g., Pang et al., 1990, Nature 343: 85; Raeymaekers, 1993, Analytical Biochem. 214: 582). Real-time amplification and detection methods generally provide a quantitative analysis before amplification products are present at high concentrations in the reaction mixture, which may be more accurate than measuring the end-point product.

Quantitative amplification methods often require that the amplification signal obtained for the amplified target sequence made from an unknown initial amount of target in a sample be compared to the amplification signal obtained for an external or internal standard (e.g., U.S. Pat. No. 5,736,333, Livak et al., and U.S. Pat. No. 6,312,929, McMillan). When comparison is to an external standard, amplification reactions are performed separately on known amounts of a standard sequence under the same conditions used for the unknown target amount. When comparison is to an internal standard (which may be referred to as an internal control, calibrator, or reference), a known amount of the standard is amplified in the same reaction with amplification of the unknown target amount. Usually, such methods presume that the amplification kinetics and efficiencies are the same for both the target and the external or internal standard which are compared (e.g., Raeymaekers, 1995, Genome Res. 5:91; Haberhausen et al., 1998, J. Clin. Microbiol. 36(3): 628, U.S. Pat. No. 5,789,153, Falkner et al., U.S. Pat. No. 5,840,487, Nadeau et al., U.S. Pat. No. 6,534,645, McMillan, and US Pat. Application No. 2002/0058262, Sagner et al.). Some methods quantitate the amount of target present in a sample by assaying for inhibition of amplification of the target when a competitor or analog is present in the reaction (e.g., U.S. Pat. No. 5,912,145, Stanley).

Various methods have been described to quantitate the initial amount or concentration of a target sequence in a specimen based on analyses of detectable signals from amplified products of the analyte sequence during or following in vitro amplification (e.g., U.S. Pat. No. 5,834,255, van Gemen et al., U.S. Pat. No. 6,447,999, Giesen et al., U.S. Pat. No. 6,503,720, Wittwer et al., U.S. Pat. No. 6,691,041, Sagner et al., US Pat. Application Nos. US 2003/0148332, Taylor et al., US 2003/0148302, Woo et al., and US 2003/0104438, Eyre et al.). Many methods rely on algorithms that include performing mathematical calculations to estimate or determine the initial analyte concentration or amount in a reaction, where such algorithms may be performed by a computerized device to perform the analysis (e.g., U.S. Pat. No. 6,066,458, Haaland et al., U.S. Pat. No. 6,713,297, McMillan et al., US Pat. Application No. US 2003/0044826, Ward et al.).

Many mathematical calculations used to quantify an initial amount or concentration of a target sequence rely on generating substantially exponential curves or derivatives thereof from the signal data associated with or produced by amplified products. As an amplification reaction proceeds, ideally the detected signal results in a curve in which the initial intensity is at a relatively low level, followed by an exponential increase in signal that is proportionate with the amount of amplified product, followed by a plateau in signal intensity when the reaction becomes depleted of substrates and/or becomes saturated with amplified product. Some methods set a threshold value that the signal must exceed to be considered a reliable indicator of detectable amplified product for use in calculating the initial amount of the target nucleic acid in the sample (e.g., U.S. Pat. No. 6,783,934, McMillan et al., U.S. Pat. No. 6,730,501, Eyre et al., US Pat. Application Nos. US 2002/0058262, Sagner et al., US 2003/0148302, Woo et al., US2003/0148332, Taylor et al., US 2003/0044826, Ward et al., and 60/659,874, Scalese et al., filed Mar. 10, 2005). Threshold-based methods seek to avoid spurious signals that may not accurately indicate nucleic acid amplification, sometimes referred to as background noise. When the detected signal data produced from an amplification assay does not result in a well-defined curve with a relatively flat beginning phase followed by an exponential phase and ending at a plateau, an analytical algorithm may produce an incorrect estimate of the initial quantity or concentration of the target analyte. For example, when the signal data varies irregularly during an amplification reaction, a method that relies on determining the point when the signal emerges above a threshold to indicate the beginning of the exponential phase of amplification may become ineffective or inaccurate for calculating the initial amount of target nucleic acid in the reaction. Examples of irregular signal data include reactions in which a first signal is detected above the threshold value but subsequent signals are detected below the threshold value, or reactions in which multiple peaks of signals are detected, or reactions in which no exponential rise in signal is detected but the signal increases relatively steadily throughout the reaction.

There remains a need for a reliable method of evaluating signal results from nucleic acid amplification reactions, particularly for real-time analyses, to determine the quantity or concentration of initial target sequences in a tested sample, particularly when the assay detects a non-ideal series of signals. Systems and methods that include performing a Fourier Transform are disclosed which respond to this need.

### SUMMARY OF THE INVENTION

A method is disclosed for determining an initial amount of target nucleic acid in a sample that includes the steps of mixing a sample that contains at least one copy of a target nucleic acid with a mixture of reaction components for performing an in vitro nucleic acid amplification reaction to amplify a sequence in the target nucleic acid, amplifying the target nucleic acid sequence in an in vitro nucleic acid amplification reaction to produce amplified products from the target nucleic acid, detecting a plurality of signals associated with the amplified products from the target nucleic acid produced during the in vitro amplification reaction, in which a characteristic of each signal provides a measurement of the quantity of the amplified products from the target nucleic acid present in the amplification reaction when each signal is detected; processing data that includes the plurality of signals associated with the amplified products from the target nucleic acid detected during the amplification reaction by performing at least one Fourier Transform calculation on the data to obtain a result; and determining an initial amount of the target nucleic acid in the sample from the result obtained in the processing step by comparing it to a calibration curve. In one embodiment, the signals associated with the amplified products are detected in a real-time amplification reaction by detecting a signal from a dye or labeled probe that binds to the amplified products. In some embodiments, the in vitro nucleic acid amplification reaction is performed by using thermocycling conditions, or by using substantially isothermal conditions. In one embodiment, detecting the plurality of signals is performed by measuring intensity of each signal at a plurality of predetermined time points or time intervals during the amplification reaction. In a preferred embodiment, the in vitro nucleic acid amplification reaction includes an internal control nucleic acid that is amplified in the same reaction mixture in which the target nucleic acid is amplified to produce amplified products from the internal control, and at least one signal specifically associated with the amplified products from the internal control is detected. Another embodiment includes processing data from signals associated with the amplified products from the target nucleic acid and processing data from detecting signals from the amplified products from the internal control. The method may also include formatting the data obtained in the detecting step into a format that is loaded into a device that performs a calculation in the processing step. In one embodiment, processing the data also includes normalizing the data to make a minimum signal value equal to about 0 and a maximum signal value equal to about 1, so that a waveform determined from the signal values spans a range from about 0 to 1. In another embodiment, the processing step includes examining the data to detect a subset of data associated with reaction mixtures in which no amplification of the target nucleic acid has occurred and removing the subset of data from further processing. In a preferred embodiment, processing the data also includes a step to optimize the data by analyzing multiple subsets of the data by performing a Fourier Transform calculation on each of the subsets to determine a portion of the data that gives optimal results. In another preferred embodiment, the processing step includes specifying a portion of the data to be used to calculate a calibration curve. In another preferred embodiment, the processing step includes both optimizing the data by analyzing multiple subsets of the data by performing a Fourier Transform calculation on each of the subsets to determine a portion of the data that gives optimal results for signals associated with the amplification products from the target nucleic acid and specifying a portion of the data to be used to calculate a calibration curve. In preferred embodiments, the processing step includes performing a Fast Fourier Transform (FFT) calculation. In one embodiment, processing the data includes calculating a gradient between a first Principle Fourier Component Used (PFCU) and a second PFCU and generating a calibration curve to which the first PFCU and second PFCU values are fitted. In another embodiment, the processing step includes performing an analysis of the data to remove subsets of the data that are considered outliers, in which outliers are values outside of a predetermined normal range of expected data. In another embodiment, determining the initial amount of the target nucleic acid in the sample includes generating a graph from processed data, from which the initial amount of target nucleic acid is calculated. Preferred embodiments use a computerized system to performing the method steps.

Another method is disclosed that calculates an initial amount of target nucleic acid in a sample, by including the steps of obtaining a data set from an in vitro nucleic acid amplification reaction in which a plurality of signals associated with amplified products from a target nucleic are detected, in which each signal provides a measurement of the quantity of the amplified products from the target nucleic acid present in the reaction at time points or time intervals during the reaction and processing the data set using a method that includes the steps of supplying information on at least one condition that characterizes the data set to be analyzed, selecting a processing option for analysis of the data set from the group consisting of (i) Blind Sample option, in which a calibration curve and processing window size are known, (ii) Fixed Window option, in which different data sets are compared under the same processing conditions but where a calibration curve is not calculated, and (iii) Optimize option, in which an efficient data window from which to calculate a calibration curve is selected, providing additional information related to computational steps performed in the processing option chosen, including a cut-off level used to determine whether amplification has taken place in a reaction and to remove from further analysis any data subset that does not provide a signal above the cut-off level, scanning the data set to determine the number of waveforms to be processed, selecting levels that are used in calculating a calibration curve, normalizing waveforms so that an initial minimal signal value is set at approximately 0 and a maximal signal value is set at approximately 1, then determining for each waveform a first data point number where a growth curve demonstrates maximal emergence to a first predetermined percentage above baseline and a second data point number where the growth curve demonstrates minimal emergence to a second predetermined percentage above baseline, to determine a data subset in a designated percentage above baseline that will be excluded from a Fourier Transform calculation, then performing the Fourier Transform calculation on a data subset of each normalized waveform that does not include excluded an data subset as determined in the previous step, calculating for each waveform one or more Principle Fourier Component Used (PFCU) values, and fitting the PFCU value for each waveform analyzed to a calibration plot to determine a calculated starting concentration based on the calibration plot, thereby determining an initial amount of the target nucleic acid in an assayed sample. In one embodiment, the method also includes a step that calculates a difference between the calculated starting concentration and the actual starting concentration, and removes data that is determined to be outlier data, in which outlier data occurs outside of a predetermined acceptable range of data. In another embodiment, the method also includes formatting the data set before the processing step to place data into a format that is used by a device that performs calculations in the processing step. In a preferred embodiment, the processing steps are scripted into a software program that is used in conjunction with a computerized device or system.

A system is disclosed for determining an initial amount of target nucleic acid in a sample, that includes a means for obtaining a data set of signals from one or more an in vitro nucleic acid amplification reactions performed by using samples that contain a target nucleic acid, in which the signals provide a measurement of amplified products for the target nucleic acid at a plurality of time points or time intervals during each reaction, a means for processing the data set that includes calculating at least one Fourier Transform of the data set or a subset of data in the data set which represents signals obtained at time points or time intervals for each reaction in which amplification of the target nucleic acid was detected, and a means for reporting a result obtained from the processed data set or subset that determines an initial amount of target nucleic acid in a sample for a reaction in which amplification was detected.

The accompanying drawings, which constitute a part of the specification, illustrate some embodiments of the invention. The figures and description explain and illustrate principles of the invention.

### BRIEF DESCRIPTION OF THE DRAWINGS

FIG. 1 is a schematic flow chart that shows steps, beginning at the top, of an algorithm embodiment that includes performing Fourier transform steps (center column, boxes 10 and 15 from the top) to produce exported results to quantitate the initial concentration or amount of analyte in a sample subjected to nucleic acid amplification.

FIG. 2 is a series of graphs (FIG. 2A to FIG. 2E) that show results obtained from nucleic acid amplification reactions that contained known amounts of a target nucleic acid that were subjected to the Fourier Transform based algorithm to determine the calculated log copy of the target nucleic acid in samples compared to the actual log copy of the target nucleic acid in the samples.

### DETAILED DESCRIPTION OF THE INVENTION

Real-time in vitro nucleic acid amplification techniques potentially have the advantage of allowing quantification over a wider dynamic range compared to end-point analyses of nucleic acid amplification assays. Many current algorithms to analyze real-time amplification data rely on the ability to fit signal data obtained from an amplification reaction to an emergence curve. That is, the algorithms produce a graphic display of the data that indicates when amplification begins by determining when a signal meets or exceeds a predetermined threshold value to indicate the beginning of exponential phase amplification. This result may be compared to an external calibration curve to quantitate the result. An inherent disadvantage of these techniques is that the real-time signal is only detectable once it emerges above the threshold value which may be difficult to determine when background signals contain “noise”, i.e., when spurious signal data points occur near or above the threshold value. Further, such algorithms often use curve-fitting techniques that use only a small number of data points close to the emergence level (above the threshold value) which may exacerbate problems associated with data that includes noise. The end result of such analyses generates a fixed point above the background signal.

Methods described herein overcome many of these problems by using data analyses that include calculation of a Fourier Transform, which for speed and ease of use may be a Fast Fourier Transform (D. F. Elliot and K. R. Rao, Fast Transforms: Algorithms, Analyses, Applications. New York: Academic Press, 1982; H. J. Nussbaumer, Fast Fourier Transform and Convolution Algorithms. New York: Springer-Verlag, 1982; A. Papoulis, Signal Analysis. New York: McGraw-Hill Book Company, 1977; L. R. Rabiner and B. Gold, Theory and Application of Digital Signal Processing. Englewood Cliffs, N.J.: Prentice-Hall, 1975; and W. H. Press, B. P. Flannery, S. A. Teukolsky, and W. T. Vetterling, Numerical Recipes in C: The Art of Scientific Computing. Cambridge: Cambridge University Press, 1988). In its most common use, the Fourier Transform de-convolutes a periodic signal in the time domain into a series of sine waves in the frequency domain. The sum of the sine waves is approximately equal to the signal in the time domain. In other words, the Fourier Transformation of a time domain signal is a representation of the corresponding frequency components that fundamentally comprise the signal.

An algorithm that is based solely in the time domain will inherently be susceptible to noise in the detected signal, i.e., spurious results that affect detection of the true signal from the assay. Noise can occur throughout the whole data set or can be apparent as a difference in baseline noise before the true signal emerges from the assay. Also, variations in end-point levels of amplification reactions can cause time-domain algorithms to be inaccurate. Data that includes results obtained from replicate tests of the same sample (e.g., an average of the results) may minimize the contributions of noise but the problems are still significant. In contrast, an algorithm that uses a Fourier Transform provides a solution to these problems because the Fourier Transform, in essence, decomposes or separates a waveform or function into sinusoids or different frequency, which sum to the original waveform. A Fourier Transform identifies or distinguishes the different frequency sinusoids and their respective amplitudes (Brigham, E. O., 1988, The Fast Fourier Transform and Its Applications, Prentice Hall, Inc. (Englewood Cliffs, N.J.)).

A Fourier Transform of data is a series of complex numbers (in the form of x+yi). Taking a power spectrum, i.e., a measurement of the power at various frequencies which is achieved by multiplying the data by its complex conjugate, yields the useful data for further analysis. For analysis of real-time nucleic acid amplification data, a practical benefit of using a Fourier Transform based algorithm is that the noise is transformed into higher frequencies compared to the dominant frequency response resulting from the signal that shows exponential amplification in the response curve. That is, inclusion of a Fourier Transform in the algorithm distinguishes noise from a true amplification signal. Using Fourier Transform based methods described herein, the gradient for replicate reactions, performed on samples in which a known initial amount of target nucleic acid was amplified, calculated between the first and second data points in the frequency domain was proportional to the number of copies of the target nucleic acid in the sample. The calculated initial amount of target nucleic acid obtained from the data by using the Fourier Transform based method was less sensitive to noise, initial baseline variability, and end-point levels of amplified sequences when the same data was analyzed by using a method that did not include a Fourier Transform.

The Fourier Transform based algorithm described herein provides advantages compared to other methods of analyzing nucleic acid amplification data. First, in the Fourier Transform based methods, there is no reliance on the signal emerging from a background detectable limit, unlike known methods that rely on the signal exceeding a predetermined threshold level. The Fourier Transform based methods provide an accurate data analysis because the calculated data using the Fourier Transform predicts the whole amplification curve, not merely a part of the curve that is detected above a background signal level. The Fourier Transform based methods do not rely only on when the signal emerges above a threshold value. Therefore, any noise in the background signal has a much smaller effect on the fitting of the signal data to a curve that is used in the Fourier Transform based method. Second, the curve fitting that the Fourier Transform based method performs is based upon a larger portion of the amplification signal compared to other methods that limit curve fitting to a portion of the data or signal in a predetermined range. Third, noise in the amplification signal itself can adversely affect the curve fitting step that is used in some algorithms that use standard methods to generate a best-fit curve from discrete signal data points. In the Fourier Transform, noise is fitted as a high frequency component of the signal. Only the first two frequency points are used by the Fourier Transform algorithm, which points are referred to as the “Principal Fourier Components Used” (PFCU), which is advantageous for systems that may be prone to more noisy data. Fourth, in contrast to existing algorithms that analyze data to determine a time of emergence above a threshold value, the Fourier Transform algorithm described herein analyzes the data both in terms of time and shape of the amplification curve. Whereas two curves that have the same emergence time but different kinetics generally would not be distinguished by algorithms that simply calculate a time of emergence relative to a threshold value, but such different data sets would be distinguished by the Fourier Transform based method described herein. The Fourier Transform based method produces an improved linear relationship, both at low and high copy levels of the initial target nucleic acid, between calculated (log) copy levels and known (log) copy levels over a range analyte concentrations. This is particularly useful for analyzing many samples in which the initial analyte concentration is in the low range where many different amplification growth curves may be seen for essentially the same amplification conditions and starting analyte levels (sometimes referred to as “fanning” because the growth curve lines do not superimpose for multiple reaction results which results in a series of lines that look like a fan when all of the reaction results are graphed together). The Fourier Transform based method is particularly useful for fitting lower copy levels where fanning of the amplification curve results of multiple reactions occurs. Fifth, the Fourier Transform based method may be used with a variety of amplification assay formats, e.g., data generated from micro-arrays as well as from individual samples that are amplified in standard test tubes or similar reaction vessels. For example, for analysis of amplification data generated in a micro-array format (which is typically noisy) by using the Fourier Transform based method, histogram analysis of detected fluorescence from the micro-array is used to generate the amplification curve. The Fourier Transform algorithm described herein was able to analyze the micro-array data and generate a good linear fit between the calculated copy levels and known copy levels of initial analyte in the amplification reactions. Finally, although the Fourier Transform algorithm describe herein has been described for use in analyzing data obtained from in vitro nucleic acid amplification reactions, the steps of the method may be useful in other applications that analyze non-amplification data types, such as in methods for detecting other molecular analytes (e.g., signal used to detect a protein analyte by using a labeled probe, such as described in EP Pat. No. 0478626, Batmanghelich et al.).

To aid in understanding preferred embodiments, some terms are defined herein. Unless stated otherwise, all scientific and technical terms used herein have the same meaning as commonly understood by those skilled in the relevant art, which may be found in the cited references or in other standard technical literature. It will be understood by those skilled in the art of molecular biology that the Fourier Transform based methods of analysis described herein do not rely on any particular type of nucleic acid amplification, or particular target analyte or control sequences, or particular primer or probe sequences. Those of normal skill in the art can design and perform an amplification reaction for an analyte of interest and apply the Fourier Transform based methods to analyze the resulting data.

“Sample” or “specimen” refers to any mixture that may contain a nucleic acid, e.g., DNA, RNA or a mixture of DNA and RNA, or analogs thereof, or cells or microorganisms that contain nucleic acids. A sample may be a tissue or material derived from a living or dead organism, such as a human, which may contain nucleic acid, e.g., sputum, blood, plasma, serum, tissue samples including swab or biopsy tissue, exudates, urine, feces, semen or other body fluids, or materials that may contain biological material. Samples may be water, soil, physical materials containing or contaminated with tissue, cells, fluids or exudates, such as may occur in environmental specimens or forensic evidence. A sample may be treated to physically or mechanically disrupt tissue or cell structure, to release intracellular components that include nucleic acids into a solution which may contain enzymes, buffers, salts, detergents and the like, such as are used to prepare, by using well known methods, nucleic acids for further analysis.

“Nucleic acid” refers to a multimeric compound comprising nucleosides or nucleoside analogs which have nitrogenous heterocyclic bases, or base analogs, linked by phosphodiester bonds or other linkages to form a polynucleotide, which may be a long polymer or shorter oligomer. The term includes conventional RNA and DNA, polymers that contain one or more DNA or RNA analogs, e.g., peptide nucleic acids (“PNA”, PCT No. WO 95/32305, Hydig-Hielsen et al.) and locked nucleic acids (“LNA”, Vester et al., 2004, Biochemistry 43(42):13233-41). A “backbone” refers to linkages, e.g., sugar-phosphodiester linkages, peptide-nucleic acid bonds, phosphorothioate or methylphosphonate linkages, or combinations of such linkages in a single polymer. The sugar moieties may be ribose, deoxyribose, or similar compounds having known substitutions, e.g., 2′ methoxy or 2′ halide substitutions. Bases may be conventional bases (A, G, C, T, U), analogs such as inosine (The Biochemistry of the Nucleic Acids 5-36, Adams et al., ed., 11^{th} ed., 1992), purine or pyrimidine base derivatives (e.g., U.S. Pat. No. 5,378,825 and PCT No. WO 93/13121), and “abasic” residues in which the backbone includes no base at one or more residues (U.S. Pat. No. 5,585,481, Arnold et al.). A nucleic acid may comprise only conventional sugars, bases and linkages of RNA or DNA, or may include conventional components and substitutions (e.g., conventional bases linked by a modified backbone, or a combination of conventional bases and base analogs).

“Oligonucleotide” or “oligomer” refers to a nucleic acid of usually less than 1,000 residues, e.g., in a size range with a lower limit of about 2 to 5 residues and an upper limit of about 500 to 900 residues, which may be purified from naturally occurring sources or made synthetically.

“Amplification oligomer” or “primer” refers to an oligonucleotide that hybridizes to a target nucleic acid, or its complement, and participates in a nucleic acid amplification reaction. Primers and promoter-primers are well known examples that generally contain at least 10 contiguous bases that are complementary to the target sequence, and optionally may include other sequences, e.g., promoter or restriction endonuclease recognition sequence. Generally, a primer oligomer can hybridize to a template nucleic acid and has a 3′ end that is extended in a nucleic acid polymerization, but may include additional sequences, e.g., 5′ promoter sequence. Any oligomer that can function as a primer can be modified to include a 5′ promoter to function as a promoter-primer, and any promoter-primer can function as a primer independent of its promoter sequence.

“Nucleic acid amplification” refers to a procedure for obtaining multiple copies of a target nucleic acid sequence, its complement, or fragments thereof (i.e, less than the complete target nucleic acid sequence or its complement). Known amplification methods include, e.g., transcription-associated amplification, e.g., nucleic acid sequence based amplification (NASBA) and transcription-mediated amplification (TMA), replicase-mediated amplification, the polymerase chain reaction (PCR), ligase chain reaction (LCR), and strand-displacement amplification (SDA) (e.g., U.S. Pat. No. 4,786,600, Kramer et al., U.S. Pat. Nos. 4,683,195, 4,683,202, and 4,800,159, Mullis et al., U.S. Pat. No. 5,422,252, Walker et al., U.S. Pat. No. 5,840487, Nadeau et al., U.S. Pat. Nos. 5,399,491 and 5,554,516, Kacian et al., U.S. Pat. No. 5,437,990, Burg et al., U.S. Pat. No. 5,130,238, Malek et al., U.S. Pat. Nos. 4,868,105 and 5,124,246, Urdea et al., and U.S. Pat. No. 5,834,255, van Gemen et al., and PCR Protocols, A Guide to Methods and Applications, Innis et al., eds., 1990). Transcription-associated amplification embodiments use substantially isothermal conditions and an RNA polymerase to produce multiple RNA transcripts from a nucleic acid template by using a promoter-primer, a primer, an RNA polymerase, a DNA polymerase, deoxyribonucleoside triphosphates (dNTP), ribonucleoside triphosphates (rNTP), and a promoter-template complementary oligonucleotide, and optionally may also include other oligonucleotides. PCR or RT-PCR amplification methods use thermocycling conditions and a first primer for amplifying one strand of the target nucleic acid, a second primer for amplifying the complementary strand of the target nucleic acid, DNA polymerase, dNTP substrates, and other components.

“Probe” refers to a nucleic acid oligomer that hybridizes specifically to a target sequence in a nucleic acid (e.g., an amplified sequence) under conditions that promote hybridization, to detect the target nucleic acid. Detection may either be direct (i.e., a probe hybridizes directly to the target) or indirect (i.e., a probe hybridizes to an intermediate molecular structure that links the probe to the target). A probe's target refers to a sequence within an amplified sequence that hybridizes specifically to the probe by using standard base pairing or some other specific binding reaction. Preferred probes for real time amplification detection include “molecular beacon” or “molecular switch” probes (e.g., U.S. Pat. Nos. 5,118,801 and 5,312,728, Lizardi et al., U.S. Pat. Nos. 5,925,517 and 6,150,097, Tyagi et al., Giesendorf et al., 1998, Clin. Chem. 44(3):482-6) and “molecular torch” probes (e.g., U.S. Pat. Nos. 6,835,542 and 6,849,412, Becker et al.). Generally, such probes include a reporter dye attached to one end of the probe oligomer (e.g., FAM™, TET™, JOE™, VIC™) and a quencher compound (e.g., TAMRA™ or Dabcyl, or a non-fluorescent quencher) attached to the other end of the probe, and signal production depends on whether the two ends with their attached compounds are in close proximity or separated. Sequences are “sufficiently complementary” if they allow stable hybridization of an oligomer to its target sequence even if the two sequences are not completely complementary. For example, a contiguous sequence that hybridizes to another sequence by standard hydrogen bonding between complementary bases may include one or more residues that are not complementary by standard base pairing, but the entire sequences can specifically hybridize together in appropriate conditions which are well known to those skilled in the art, can be predicted readily from sequence composition, or can be determined by using routine testing (e.g., See Sambrook et al., Molecular Cloning, A Laboratory Manual, 2^{nd} ed. (Cold Spring Harbor Laboratory Press, Cold Spring Harbor, N.Y., 1989) at §§ 1.90-1.91, 7.37-7.57, 9.47-9.51 and 11.47-11.57 particularly at §§ 9.50-9.51, 11.12-11.13, 11.45-11.47 and 11.55-11.57).

Assays may include sample or specimen processing to separate, purify, isolate or concentrate nucleic acids in general or the target nucleic acid specifically, by using methods that are well known to those skilled in the art (e.g., U.S. Pat. Nos. 6,110,678, 6,280,952, and 6,534,273, Weisburg et al., Boom et al., 1990, J. Clin. Microbiol. 28(3): 495, and PCT No. WO 03/046177, Baker et al.).

Although preferred embodiments described herein detect signals resulting from fluorescent compounds and analyze the data in the Fourier transform based method, those skilled in the art will appreciate that any label that can be detected or lead to a detectable response may be used to produce data suitable for analysis by using the described methods. Such labels are well known and may be joined, directly or indirectly, to a nucleic acid probe or to the amplified nucleic acid for detection. For example, a dye may bind directly to an amplified nucleic acid.

A nucleic acid amplification reaction that produces data to be analyzed by using the Fourier Transform based methods may be analyzed with reference to an internal or external control or standard. Generally an external control or standard refers to a sample or series of samples, e.g., a dilution series, that contain a known amount of a nucleic acid that is treated and amplified using substantially the same conditions as the amplification reaction for the intended analyte nucleic acid, to generate a standard set of amplification signals or growth curve for comparison to the signals or curve generated when a sample containing an unknown amount of the analyte is assayed. An internal control, internal calibrator, or internal reference generally refers to a known amount of non-analyte control nucleic acid that is included in a reaction in which both the target analyte and control nucleic acids are amplified under essentially the same conditions in the same reaction. A first signal is produced from amplification of the non-analyte control sequence and a second signal, distinguishable from the first signal, is produced from amplification of the analyte target nucleic acid. An embodiment of an internal control has been described in detail previously (U.S. patent application Ser. No. 11/418,931, Marlowe et al., filed May 4, 2006).

A plurality of standard samples and at least one test sample may be assayed in which the plurality of standard samples each contain a known starting quantity of a control sequence and a known starting quantity of an analyte target sequence. The starting concentrations of the control sequence in the standard samples are preferably set to equal levels and the test sample contains a known starting quantity of the control sequence and an unknown starting quantity of the analyte target sequence. The nucleic acid sequences in the standard samples and test sample are then amplified under the same conditions during an amplification time interval by using a known in vitro amplification method, such as PCR, which uses thermocycling, or TMA, which uses substantially isothermal conditions. Assays may use two differently labeled primers (signal primers, one specific for the control sequence and one specific for the analyte target sequence) that serve as detection probes for amplification of the nucleic acids in the reactions (i.e., one for labeling control amplicons and one for labeling target amplicons). Alternatively, detection probes that do not serve as primers may be included in the reactions, i.e., differently labeled probes, that produce distinguishable signals, one specific for the control sequence that detects control amplicons, and one specific for the analyte target sequence that detects target amplicons. During the amplification reaction, each detector probe binds to its specific amplicon and is converted to a form that produces a detectable signal (e.g., a hydrolysis-resistant chemiluminescent signal or a higher fluorescence intensity than in the unconverted form). Signals are detected from the amplicons using standard methods appropriate for the label used (e.g., a luminometer that detects light at appropriate time intervals for the different luminescent compounds or a fluorometer that detects fluorescence at appropriate wavelengths for the different compounds). Signals are preferably measured in real-time during the amplification reaction, preferably at predetermined time points or intervals (which may be normalized to respective measurement time points) during the amplification reaction time interval. For example, during each of a plurality of consecutive measurement time intervals during an amplification reaction time, a plurality of signal measurements are performed on control and test samples.

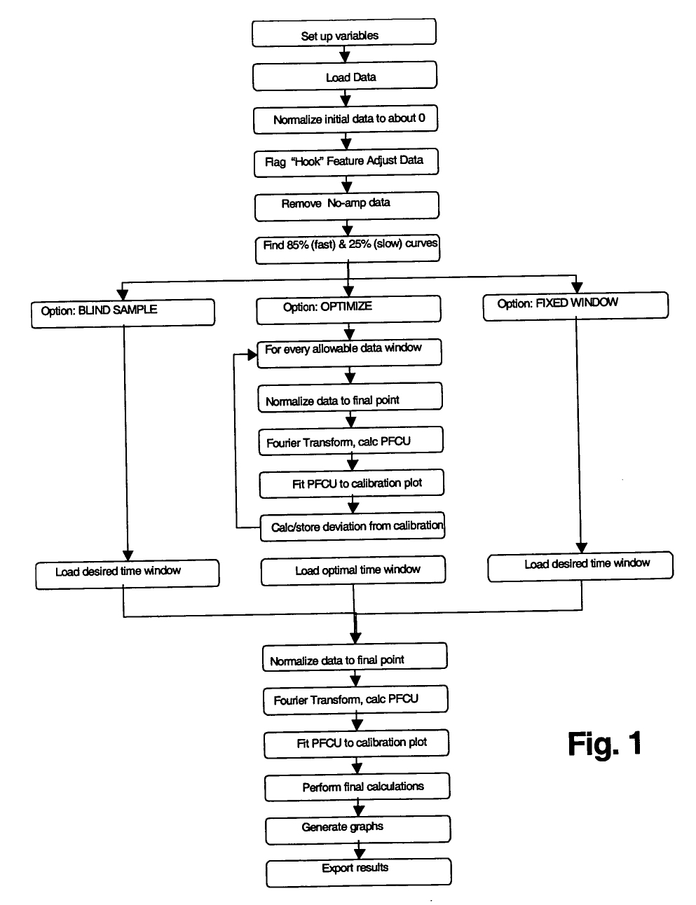

The Fourier Transform based algorithm used to analyze collected data for signal measurements over an amplification reaction time interval is summarized by the embodiment illustrated in FIG. 1. This flow-chart shows the main features of the Fourier Transform algorithm. The initial step (top box) sets up the detection device with the appropriate variables (e.g., wavelength and time intervals for signal detection). The collected signal data is then loaded into the algorithm. Individual data sets are normalized so that their background levels are approximately zero, although this is an optional step that is not required for the analysis but is useful for comparing and graphing the input data during the final analysis. The data for each waveform is examined to see if it has a characteristic kinetics, which may be referred to as a “hook and handle” feature, i.e., to establish if it fits into a characteristic response where a maximum fluorescence value is reached followed by a decline in fluorescent signal. If the waveform has this “hook” feature, it is flagged and the data will be adjusted later (at the “normalize data” step, center column, box 9), such as by removing the decline in fluorescent signal and replacing it with a straight line at the level of the maximum amplitude of the waveform. This has been shown not to affect the data quality and avoids the algorithm fitting an incorrect portion of the data. Each data set is then examined to ensure that amplification took place (e.g., a signal is detected above a predetermined background level or the data for that sample is considered to be “non-amp data”). A cut-off limit is used in the time domain to distinguish between positive amplification samples and negative amplification (or “non-amp”) samples. The subset of data that does not meet or exceed the cut-off limit (i.e., no-amp data) is removed from the data to be analyzed, and optionally a record of which waveforms are removed is kept to indicate negative samples to the user. If the user has chosen the option to “optimize” the data and calibration curve, sub-sets of the complete waveforms or “windows” are analyzed. Every allowable window (limits are set to ensure that all relevant data is included) is passed through a Fourier Transform calculation to determine which window gives optimal results. Alternatively, the user may specify the window to be used to calculate the calibration curve (this is used to calculate corresponding internal control (“IC”) channels of a data set) or may provide both the window and calibration data (for the blind samples). Within the chosen window, each waveform is normalized so that its maximum amplitude is one (i.e., each waveform spans from 0 to 1). The waveform is then transformed by performing a Fast Fourier Transform (FFT) calculation. The result of this transformation is a set of complex number frequency components. These components are then multiplied by their complex conjugate so that a non-complex representation of the magnitude of the frequency components is established. The gradient between the first two resulting frequency components (Principle Fourier Component Used or “PFCU”) is then calculated. A calibration curve is then generated from this data and the final calculated (log) copy level established from the calibration curve (“fit PFCU to calibration plot”). Results may be generated in the form of graphs, tables, reports, or any format may be used, and the results are exported for use and storage, such as in a printed document, screen display, and/or electronic data file.

One embodiment includes additional steps in the algorithm diagramed in FIG.1, which is the steps of performing an analysis of the data to remove data sets in which the assay provided signal that are considered “outliers”, i.e., data that falls above or below a predetermined value such that the data is considered to represent unreliable results, such as might occur if a reagent or device used in the assay were contaminated or malfunctioning. A “perform outlier analysis” step is typically inserted between the “Fourier Transform, calc PFCU” and “Fit PFCU to calibration plot” steps that appear twice in FIG. 1 (center column, boxes 10 to 11 and 15 to 16).

Preferred embodiments of the method use instructions in a computer program to efficiently perform the algorithm steps. Such embodiments may be performed by using a computerized device that performs steps in a script contained in computer software. For example, a software product may be programmed to perform the steps.

In one preferred embodiment the algorithm is coded in a commercially available MATLAB® technical computing platform (The MathWorks, Inc., Natick, Mass.), which can be exported subsequently to run as a standalone executable program, e.g., in a WINDOWS® operating system (Microsoft Corp., Redmond, Wash.). Briefly, the algorithm in the MATLAB® platform performs the following steps. (1) Initial variables that will be used throughout the algorithm are defined and reserved. This platform is based on matrix mathematics which allows multiple waveforms (i.e. results of multiple amplifications with the same conditions) to analyzed at the same time. (2) Experimental results in a predefined format are loaded into the MATLAB® environment. Before performing the algorithm, typically a separate task (described below) is performed to format the data obtained from fluorescent reader devices into a form that is useful for the device and software that performs the computational tasks of the algorithm. Because many real-time fluorescence readers record multiple fluorescent channels, the user provides information on which channel to use and the appropriate data set is selected. The data set that is loaded can contain up to six different experimental conditions with up to twelve waveforms for each condition. In a preferred embodiment, six different conditions and up to twelve waveforms per condition are chosen as maximum, but those skilled in the art will understand that these numbers can be increased or decreased as desired, with additional standard programming. In this embodiment, each condition is analyzed separately and the user is prompted to choose which condition is to be processed, although those skilled in the art will realize that the algorithm may be expanded to process multiple conditions if required. (3) Once the correct data set (e.g., condition, fluorescent channel) is loaded, the algorithm calculates a number of parameters based on data size that will be used throughout the rest of the algorithm. (4) The user is prompted to select one of three processing options: (i) Blind Sample, where calibration curve and processing window size are known, (ii) Fixed Window, which allows different data sets to be compared under the same processing conditions but where a calibration curve is not calculated, or (iii) Optimize, which is used with data sets of known starting copy levels and used to select automatically the most efficient data window from which to calculate a calibration curve. Any of steps 5, 6, and 7 are performed, based on which of the processing options is chosen. (5) When the Blind Sample option is chosen, the user is prompted to enter calibration, processing window and no-amp cut-off level information (e.g., Calibration A, Calibration B, Start Data Point, End Data Point, No-Amp cut off). Calibration A and Calibration B are used to calculate the calibration curve in the form of a linear relationship:
Y=(Calibration A)×+(Calibration B).
The Start Data Point and End Data Point define the range of data within the whole data set that will be used for processing. The No-Amp cut off is a value used to determine whether amplification has taken place and waveforms that do not have a fluorescent response above this value are considered to be non-amplified. (6) When the Fixed Window option is chosen, the user is prompted to enter processing window and no-amp cut-off level information (Start Data Point, End Data Point, No-Amp cut off level). The Start Data Point and End Data Point define the range of data within the whole data set that will be used for processing. The No-Amp cut off is a value used to determine whether amplification has taken place and waveforms that do not have a fluorescent response above this level are considered to be non-amplified. (7) When the Optimize option is chosen, the user is prompted to enter no-amp cut-off level information (No-Amp cut off level), which is a value used to determine whether amplification has taken place, and waveforms that do not have a fluorescent response above this level are considered to be non-amplified. (8) Following step 5, 6 or 7, the data set is scanned to determine the number of waveforms to be processed (e.g., up to twelve) and positions for which there is no data for a particular waveform are recorded (e.g., if only six waveforms are used in a particular condition matrix lines 7 through 12 will be noted as containing no data). (9) The pre-loaded data also may contain the starting concentration levels of each data set (but this option is not given if the Blind Sample processing option is chosen). This data is presented to the user, who may select which levels are used in calculating calibration curves (e.g., the user may choose to exclude copy levels that are above or below the expected range of a linear calibration curve). (10) The waveforms are then normalized at the start of the data point so that their initial fluorescent values are approximately zero. This step is not needed for the Fourier Transformation but is included in some preferred embodiments because normalized (low end) data is more easily graphed at the final step. In this embodiment, the mean of the first eight data points are used to normalize against, but those skilled in the art will realize that other data may be used, e.g., a different number and/or location of the data points. (11) A determination is made for each waveform to establish if it fits into a characteristic response where a maximum fluorescence value is reached followed by a decline in fluorescent signal (which may be referred to as a “hook and handle” feature). If the determination is made, then the waveform is flagged and the decline in fluorescent signal is removed during the normalization to 1 task (step 13.1.1). Removing the characteristic and replacing it with a maximum value of 1 has been shown not to affect data quality and eliminates the chance of the algorithm choosing a data window that comprises solely of the post-maximum decline. (12) For each waveform, the data point number where the fastest emerging curve has emerged to a certain percentage above background and the data point number where the slowest emerging curve has emerged to a certain percentage above background are determined. These two values are used as boundary conditions in the optimization routine. This allows a simple percentage above baseline determination to be used as the actual figure, which, even if subject to noise, will not be used in the Fourier Transformation. (13) If the Optimize option was chosen, then steps 13.1 (including 13.1.1 to 13.1.8) to 13.2 are performed. (13.1) A loop is set up that varies the length and location of a window of the data points (with boundary conditions) within the complete data set that is used in data analysis. For each of these possible windows:
(13.1.1) Each waveform is normalized to have a maximum value of 1. Therefore, each waveform is now a set of values between 0 and 1. (13.1.2) A Fast Fourier Transform is calculated for each waveform (i.e., MATLAB® fft command). This computation returns the discrete Fourier Transform (DFT) of vector X, computed with a Fast Fourier Transform (FFT) algorithm of the form

$$X ( k ) = {\sum }_{j = 1}^{N} x ( j ) {ω}_{N}^{( j - 1 ) ( k - 1 )}$$

is an N^{th} root of unity.
(13.1.3) Each resulting data-point is then multiplied by its complex conjugate to give a representation of the power spectrum of the transformed data (the resulting data is in the real domain only). (13.1.4) The gradient, or difference (as data spacing is 1) between the first and second points of this resulting series is calculated for each waveform, and this value is referred to as the Principle Fourier Component Used (PFCU). (13.1.5) A linear calibration plot is constructed from the PFCU for each waveform and its known starting concentration level (using the starting concentration levels chosen previously). In other embodiments, a non-linear calibration curve may be implemented. (13.1.6) For each waveform, a computed starting concentration level, based on the calibration curve above, is calculated and the difference between the calculated and actual starting concentration is determined. (13.1.7) Outliers are removed, which in this embodiment is based on standard deviation levels, but other methods of removing outlier data can be used as will be appreciated by those skilled in the art. (13.1.8) A total error for the curve is calculated and the calibration values Calibration A and Calibration B (defining the calibration curve) are recorded with the time window start and end points of the particular time window in the loop.
   (13.2) The window of data points that contains the least errors (i.e. best fit to the calibration curve) is chosen and the start and end data point numbers recorded as the optimal setting along with the corresponding calibration data (Calibration A and Calibration B). (14) For all of the available options, there is now a known time window start and end point. For the Optimize and Blind Sample options, there are now known values of Calibration A and Calibration B that define the calibration curve. (15) For this defined time window, steps 13.1.1 to 13.1.4 are repeated. (16) If the Optimize or Blind Sample options have been chosen, a calibration curve is constructed from the known Calibration A and Calibration B values and the PFCU data fitted to this calibration curve. Calculated starting concentrations are calculated from the data points and curve. (17) If the Fixed Window option was chosen, only the PFCU values are calculated. (18) The algorithm then provides the user with a readout of the analyzed data, e.g., it plots one or more graphs and/or exports the analyzed data (e.g., to a spreadsheet, such as EXCEL® (Microsoft Corp.)).

In some embodiments, the method may also include use of additional tools to appropriately format signal data obtained from a detection device and transfer the formatted data to a device for performing the Fourier Transform-based algorithm. A preferred embodiment uses a computerized tool, such as an EXCEL® macro attached to a spreadsheet, but those skilled in the art will appreciate that the reformatting and transferring functions may be performed using a variety of methods or tools. Data may be collected from any of a variety of amplification and signal detection devices (e.g., DNA Engine Opticon®2 Real-Time PCR or Chromo4™ detection systems (Bio-Rad Laboratories, Inc., Hercules, Calif.), ABI 7000 Real Time PCR system (Applied Biosystems Inc., Foster City, Calif.), Rotogene PCR device (Corbett Research, Mortlake, Australia), Fluoroskan Ascent® system (Thermo Electon Corp., Waltham, Mass.), and TIGRIS® system (Gen-Probe Incorporated, San Diego, Calif.).

An embodiment that includes reformatting and transferring the data uses the following steps. First, identify the source of the signal data, e.g., instrument from which the data was collected, test date, operator information, and the like. Second, identify landmarks in the original data collection (e.g., columns or rows of a data table that correspond to an array of reactions, integers that correspond to individual reactions in a multiplicity of reactions, and the like) and determine the total number of samples in the data collection. Third, sort the collected data in an organized manner that correlates the signal data with tested samples, e.g., in sequential order of data collection, in an array corresponding to the reaction array (e.g., like the order of reactions in a multi-well plate), in data groups, such as for replicate tests of a sample or correlated with a physical characteristic such as a dilution series of a sample, and the like. Fourth, construct a worklist that identifies each tested sample with its collected signal data, where each sample may be identified by a type and quantity of information selected by the user, e.g., test position, target levels present in standards, replicate tests of individual samples, sample sources or codes, and the like. Preferably, a separate worklist is constructed for each data collection, e.g., one worklist for a fluorescence range or dye signal detected for a particular test. For example, in an assay in which four individual signals or emission ranges were detected for each tested sample, four worklists are constructed with identifying information for each group of the collected data, i.e., one worklist per detected emission range or dye.

Fifth, transfer the formatted collected data in each worklist to a device or system that performs the Fourier Transform (i.e., the “load data” step of FIG. 1).

Those skilled in the art will appreciate that the Fourier Transform based algorithm described herein may be associated with one or more devices, or an apparatus or system that performs biochemical steps of an assay (e.g., dispensing of reagents into mixtures for nucleic acid amplification and detection, incubation of reaction mixtures, and detection of signals from the reaction mixtures) in addition to the data analysis steps performed by the algorithm. The Fourier Transform based algorithm may be associated as part of an integrated system (e.g., a single apparatus that performs biochemical operations, detection and data analysis steps), or may be as an add-on configured by the user (e.g., a system in which the user assembles more than one device which cumulatively include biochemical and/or detection steps with data analysis steps that include the Fourier Transform based algorithm. That is, the Fourier Transform based algorithm described herein may be included in a computer program that is used with a variety of devices, instruments or systems. A preferred embodiment is a system that include a means for performing a real-time nucleic acid amplification reaction, a means for detecting signals from the real-time amplified nucleic acids, a means for formatting the signal data, and a means for performing the Fourier Transform based algorithm. In a preferred embodiment, the Fourier Transform based algorithm is associated with a computerized integrated system that provides a response to the user that quantitates the initial amount or concentration of the analyte of interest in a tested sample.

### EXAMPLE 1

### Analysis of Amplification Data Using the Fourier Transform Algorithm

This example demonstrates analysis of data using the Fourier Transform based algorithm described above for fluorescent signals obtained during real-time nucleic acid amplification of a sample that contained a known amount of an analyte (HIV-1 RNA at 0 to 5.7 log copies per reaction) and a known amount of a synthetic internal control (synthetic RNA at 400 copies per reaction). The assay included preparation of samples containing known amounts of the analyte and internal control RNA in a substantially aqueous solution, target capture of the analyte and internal control RNA from the solution using a mixture of capture oligomers, one specific for the analyte (SEQ ID NO:4) and one specific for the internal control (SEQ ID NO:8) and magnetic particles with an immobilized oligomer (50 μg/ml) that is partially complementary to each of the capture oligomers, using methods substantially as described previously in detail (U.S. Pat. Nos. 6,110,678 and 6,280,952, Weisburg et al.). Briefly, the samples containing known amounts of the analyte and internal control RNA were mixed with the capture oligomers and immobilized oligomers in a hybridization reagent, incubated at 61° C. for 30 min, then incubated at room temperature for 30 min, the immobilized hybridization complexes on the magnetic particles were magnetically separated from the solution phase, washed twice at room temperature. The magnetic particles with the captured analyte and internal control RNAs were then mixed with amplification reagents that included primers, fluorescent compound labeled probes, amplification substrates, enzymes, salts and buffering agents in a substantially aqueous solution phase for amplification in a transcription mediated amplification reaction (substantially as described previously in detail in U.S. Pat. Nos. 5,399,491 and 5,554,516, Kacian et al., in reaction mixtures of about 0.06 ml, each reaction mixture placed in a well of a 96-well plate incubated in a Chromo4™ system device (Bio-Rad Laboratories, Inc., Hercules, Calif.)). The captured RNA were amplified by using primers specific for the analyte RNA (SEQ ID NO:1 at 0.102 pmol/μl, SEQ ID NO:2 at 0.18 pmol/μl) and for the internal control RNA (SEQ ID NO:5 at 0.167 pmol/μl, SEQ ID NO:6 at 0.034 pmol/μl) and detected by hybridization to a detection probe specific for each of the amplified products (molecular torch probe of SEQ ID NO:3 for the analyte product, at 0.267 pmol/μl, and molecular beacon probe of SEQ ID NO:7 for the internal control product at 0.5 pmol/μl). The detection probes were synthesized using RNA bases linked by 2′-O-methyl linkages, and labeled with a fluorescent compound at the 5′ end (FAM for the analyte probe, HEX for the internal control probe) and a quencher compound at the 3′ end (Dabcyl for both). The reaction mixtures containing the amplification reagents without enzymes were incubated at 60° C. for 10 min, then at 42° C. for 5 min, then the enzymes (MMLV reverse transcriptase (RT) and T7 RNA polymerase were mixed into each reaction, at about 224-248 U/μl and 100-140 U/μl, respectively; wherein 1 U of MMLV-RT incorporates 1 nmol of dTTP in 10 min at 37° C. using 200-400 micromolar oligo-dT-primed poly(A) as template and 1 U of T7 RNA polymerase incorporates 1 nmol of ATP into RNA in 1 hr at 37° C. using a DNA template containing a T7 promoter). Then the reaction mixtures were incubated at 42° C. for the remaining reaction time (about 50 min), during which fluorescent emissions were measured in 75 time intervals, for 3 sec each (in a first channel for FAM emission detection at 515-530 nm, and in a second channel for HEX emission detection at 560-580 nm), expressed as relative fluorescence units (RFU).

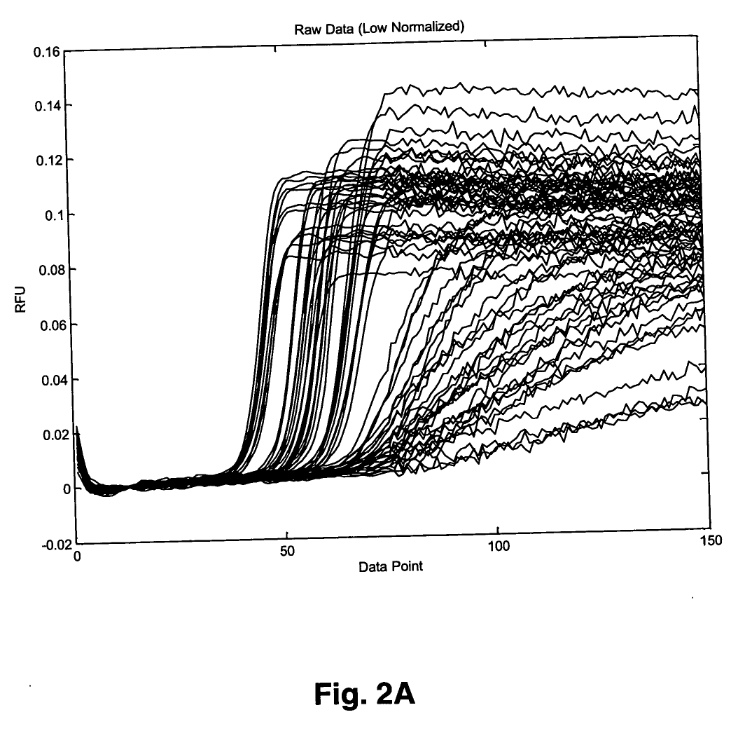

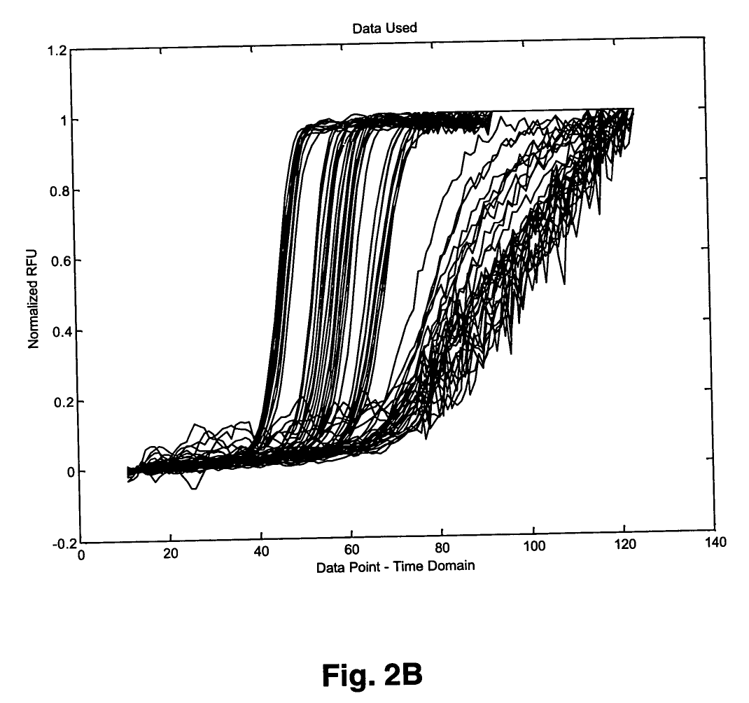

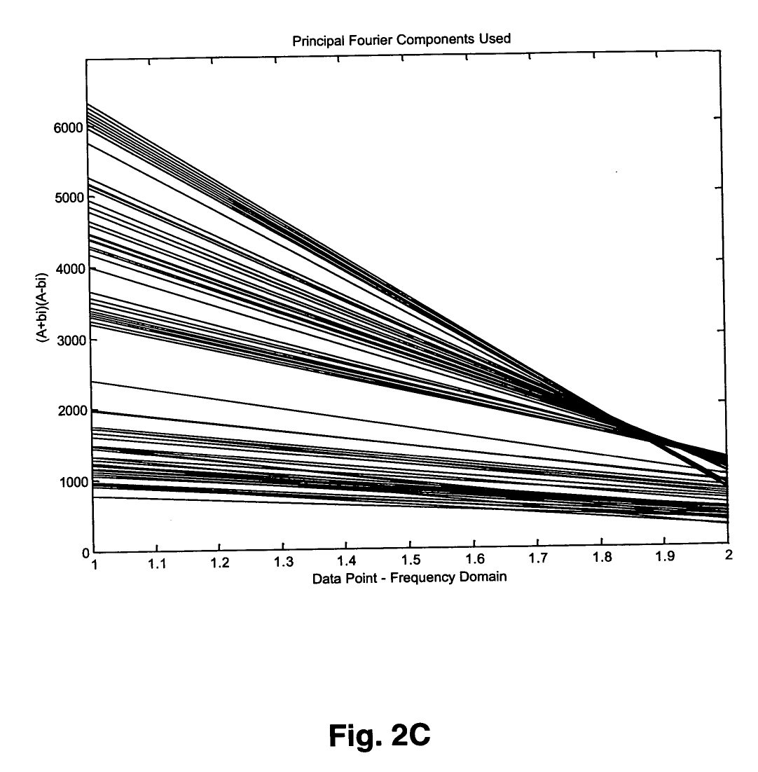

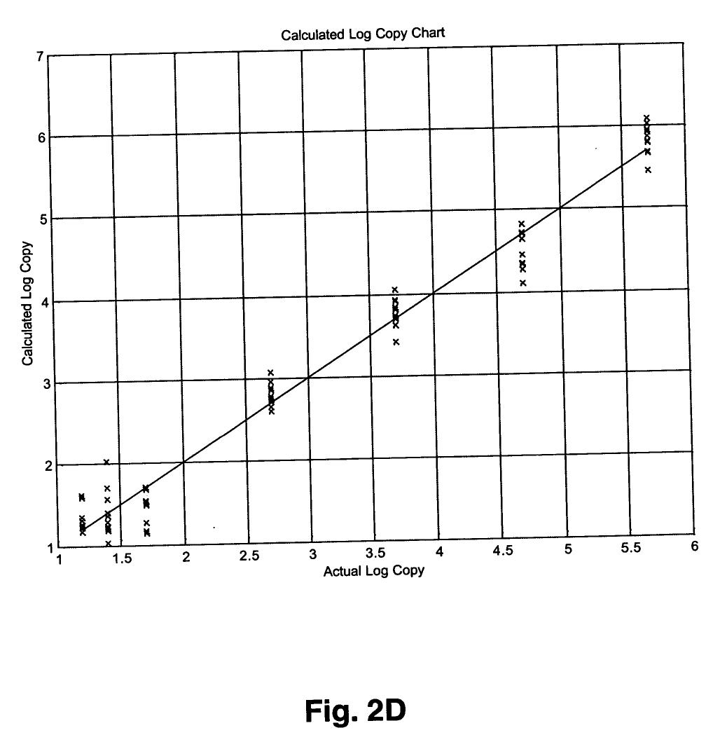

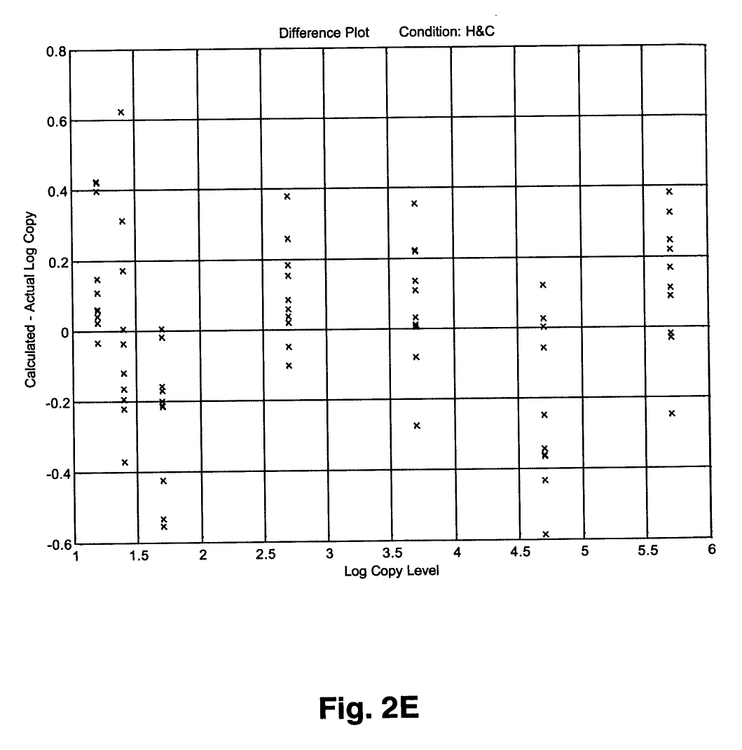

FIG. 2 shows an example of graphs produced by the Fourier Transform algorithm described above on a large number of replicate samples which contained known log copies of the analyte nucleic acid in a range of 1 to about 6. FIG. 2A shows a graph of the raw data from these assays, normalized at the low detection end so that all lines in the graph are plotted so that the detected RFU start at 0 RFU (i.e., the initial data has been normalized by setting the minimal signals detected to about 0 RFU). FIG. 2B shows a graph of data points in the time domain, used in the Fourier Transform based algorithm, where all of the detected signal curves have been normalized to fall between a minimum of 0 RFU and a maximum of 1 RFU. FIG. 2C shows a graph of the Principal Fourier Components Used (PFCU) following application of the Fourier Transform calculation applied to the data of each of the curves shown in FIG. 2B. FIG. 2D shows the results of the calibration chart in which the actual log copy (x-axis) and the calculated log copy (y-axis) are plotted for the samples for which the data was processed using the Fourier Transform based algorithm, showing a linear relationship over the actual log copy range of 1 to 5.5. FIG. 2E shows the results of a difference plot in which the actual log copy level (x-axis) and the calculated minus actual log copy level (y-axis) are plotted, to show that the deviation between actual and calculated values fell generally within ±0.5 log copies when the data was processed using the Fourier Transform based algorithm.

We claim:

1. A method of determining an initial amount of target nucleic acid in a sample, comprising the steps of:
mixing a sample that contains at least one copy of a target nucleic acid with a mixture of reaction components for performing an in vitro nucleic acid amplification reaction to amplify a sequence in the target nucleic acid;
amplifying the target nucleic acid sequence in an in vitro nucleic acid amplification reaction to produce amplified products from the target nucleic acid;
detecting a plurality of signals associated with the amplified products from the target nucleic acid produced during the in vitro amplification reaction, wherein a characteristic of each signal provides a measurement of the quantity of the amplified products from the target nucleic acid present in the amplification reaction when each signal is detected;
processing data that includes the plurality of signals associated with the amplified products from the target nucleic acid detected during the amplification reaction by performing at least one Fourier Transform calculation on the data to obtain a result; and
determining an initial amount of the target nucleic acid in the sample from the result obtained in the processing step by comparing it to a calibration curve.

2. The method of claim 1, wherein the signals associated with the amplified products are detected in a real-time amplification reaction by detecting a signal from a dye or labeled probe that binds to the amplified products.

3. The method of claim 1, wherein the in vitro nucleic acid amplification reaction is performed by using thermocycling conditions.

4. The method of claim 1, wherein the in vitro nucleic acid amplification reaction is performed by using substantially isothermal conditions.

5. The method of claim 1, wherein detecting the plurality of signals is performed by measuring intensity of each signal at a plurality of predetermined time points or time intervals during the amplification reaction.

6. The method of claim 1, wherein the in vitro nucleic acid amplification reaction includes an internal control nucleic acid that is amplified in the same reaction mixture in which the target nucleic acid is amplified to produce amplified products from the internal control, and wherein at least one signal specifically associated with the amplified products from the internal control is detected.

7. The method of claim 6, wherein processing the data that includes processing data from signals associated with the amplified products from the target nucleic acid and processing data from detecting signals from the amplified products from the internal control.

8. The method of claim 1, which further includes formatting the data obtained in the detecting step into a format that is loaded into a device that performs a calculation in the processing step.

9. The method of claim 1, wherein processing the data further includes normalizing the data to make a minimum signal value equal to about 0 and a maximum signal value equal to about 1, so that a waveform determined from the signal values spans a range from about 0 to 1.

10. The method of claim 1, wherein processing the data further includes examining the data to detect a subset of data associated with reaction mixtures in which no amplification of the target nucleic acid has occurred and removing the subset of data from further processing.

11. The method of claim 1, wherein processing the data further includes an option to optimize the data by analyzing multiple subsets of the data by performing a Fourier Transform calculation on each of the subsets to determine a portion of the data that gives optimal results.

12. The method of claim 1, wherein processing the data further includes an option to specify a portion of the data to be used to calculate a calibration curve.

13. The method of claim 1, wherein processing the data further includes an option to both optimize the data by analyzing multiple subsets of the data by performing a Fourier Transform calculation on each of the subsets to determine a portion of the data that gives optimal results for signals associated with the amplification products from the target nucleic acid and to specify a portion of the data to be used to calculate a calibration curve.

14. The method of claim 1, wherein the Fourier Transform calculation is a Fast Fourier Transform (FFT) calculation.

15. The method of claim 1, wherein processing the data further includes calculating a gradient between a first Principle Fourier Component Used (PFCU) and a second PFCU and generating a calibration curve to which the first PFCU and second PFCU values are fitted.

16. The method of claim 1, wherein processing the data further includes performing an analysis of the data to remove subsets of the data that are considered outliers, wherein outliers are values outside of a predetermined normal range of data expected from the amplifying and detecting steps.

17. The method of claim 1, wherein determining the initial amount of the target nucleic acid in the sample includes generating a graph from the processed data from which an initial amount of target nucleic acid in the sample is calculated.

18. A computerized system for performing the method of claim 1.

19. A method of calculating an initial amount of target nucleic acid in a sample, comprising the steps of:
obtaining a data set from an in vitro nucleic acid amplification reaction in which a plurality of signals associated with amplified products from a target nucleic are detected, wherein each signal provides a measurement of the quantity of the amplified products from the target nucleic acid present in the reaction at time points or time intervals during the reaction;
processing the data set by performing a method that includes the steps of:
supplying information on at least one condition that characterizes the data set to be analyzed,
selecting a processing option for analysis of the data set from the group consisting of (i) Blind Sample option, in which a calibration curve and processing window size are known, (ii) Fixed Window option, in which different data sets are compared under the same processing conditions but where a calibration curve is not calculated, and (iii) Optimize option, in which an efficient data window from which to calculate a calibration curve is selected,
providing additional information related to computational steps performed in the processing option chosen, including a cut-off level used to determine whether amplification has taken place in a reaction and to remove from further analysis any data subset that does not provide a signal above the cut-off level,
scanning the data set to determine the number of waveforms to be processed,
selecting levels that are used in calculating a calibration curve,
normalizing waveforms so that an initial minimal signal value is set at approximately 0 and a maximal signal value is set at approximately 1,
determining for each waveform, a first data point number where a growth curve demonstrates maximal emergence to a first predetermined percentage above baseline and a second data point number where the growth curve demonstrates minimal emergence to a second predetermined percentage above baseline, to determine a data subset in a designated percentage above baseline that will be excluded from a Fourier Transform calculation,
performing a Fourier Transform calculation on a data subset of each normalized waveform that does not include the data subset to be excluded from the Fourier Transform calculation determined in the previous step,
calculating for each waveform one or more Principle Fourier Component Used (PFCU) values, and
fitting the PFCU value for each waveform analyzed to a calibration plot to calculate a calculated starting concentration based on the calibration plot, thereby determining an initial amount of the target nucleic acid in an assayed sample.

20. The method of claim 19, wherein the method further includes calculating a difference between the calculated starting concentration and the actual starting concentration, and removing data that is determined to be outlier data, wherein outlier data occurs outside of a predetermined acceptable range of data.

21. The method of claim 19, wherein the method further includes formatting the data set before the processing step into a format that is used by a device that performs calculations in the processing step.

22. The method of claim 19, wherein the processing steps are scripted into a software program that is used in conjunction with a computerized device or system.

23. A system for determining an initial amount of target nucleic acid in a sample, comprising:
a means for obtaining a data set of signals from one or more an in vitro nucleic acid amplification reactions performed by using samples that contain a target nucleic acid, wherein the signals provide a measurement of amplified products for the target nucleic acid at a plurality of time points or time intervals during each amplification reaction;
a means for processing the data set that includes calculating at least one Fourier Transform of the data set or subset of data in the data set which represents signals obtained at time points or time intervals for each amplification reaction in which amplification of the target nucleic acid was detected; and
a means for reporting a result obtained from the processed data set or subset that determines an initial amount of target nucleic acid in a sample for an amplification reaction in which amplification of the target nucleic acid was detected.
